(19) United States
(12) Patent Application Publication
(10) Pub. No.: US 2022/0332915 A1
(43) Pub. Date: Oct. 20, 2022

(54) Enzymatic Degradation of Nylon

(71) Applicant: Battelle Memorial Institute, Columbus, OH (US)

(72) Inventors: Katarzyna H. Kucharzyk, Columbus, OH (US); Morgan V. Evans, Columbus, OH (US); Emma Beasley, Columbus, OH (US)

(21) Appl. No.: 17/713,220

(22) Filed: Apr. 4, 2022

Related U.S. Application Data
(60) Provisional application No. 63/170,562, filed on Apr. 4, 2021.

(51) Int. Cl. C08J 11/10; C12N 9/18

(52) U.S. Cl. CPC C08J 11/105; C12N 9/18; C12Y 301/01074; C12Y 301/01003; C08J 2333/24

(57) ABSTRACT

Novel ways of degrading polyamides including Nylon 6 and Nylon 6,6 (polycaprolactam) are disclosed. Microorganisms originally sourced from the environment are utilized to produce an enzyme(s) useful for degrading polyamides. can be degraded by a cutinase, a lipase, or a combination thereof.

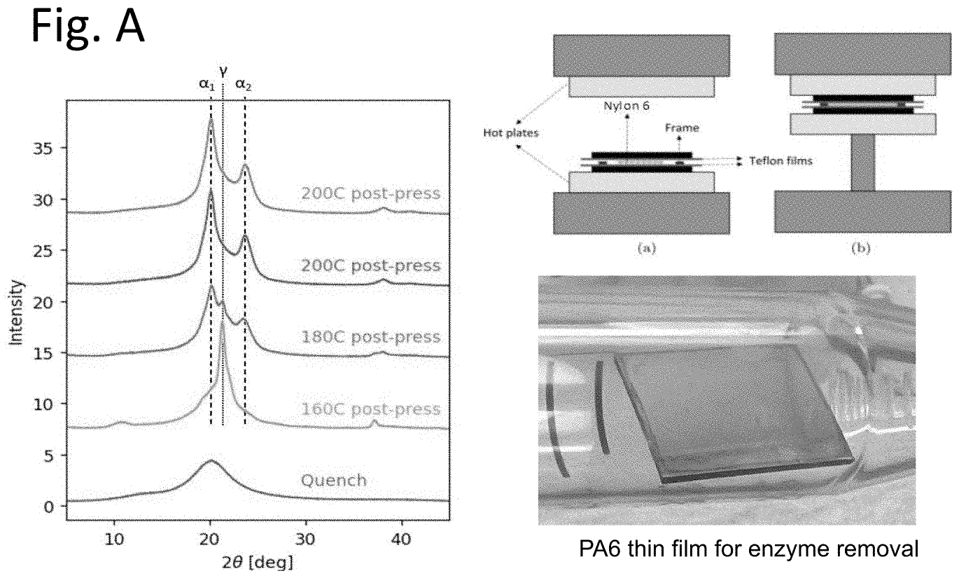

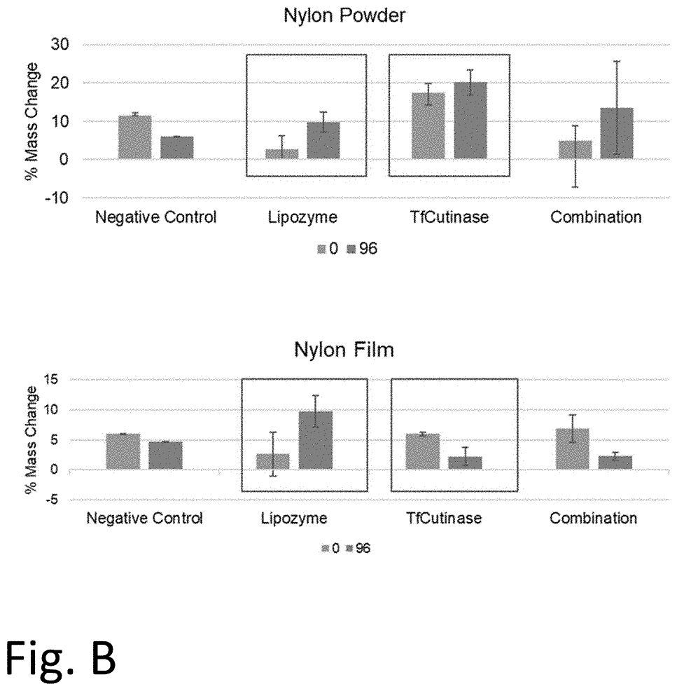

### RELATED APPLICATION

This application claims priority to U.S. Patent Application Ser. No. 63/170,562 filed Apr. 4, 2021, which is incorporated herein as if reproduced in full below.

### GOVERNMENT RIGHTS CLAUSE

This invention was made with Government support under contract 00033145 awarded by the U.S. Department of Energy. The Government has certain rights in this invention.

### INTRODUCTION

Nylon-6 (PA6) is a highly recalcitrant, synthetic polymer whose environmental and economic footprint could be reduced through enzymatic degradation and upcycling technologies. PA6 is used in bristles, ropes, fabrics, fishing nets, automobile parts, and more. PA6 has an amide backbone obtained by the ring-opening polymerization of E-caprolactam. The U.S. annual consumption of PA6 is 0.449 million metric tons (MMT/year), and 5.528 MMT/year globally.

These usages are on par with vinyl acetates and acrylics, just shy of polyurethane and rubber annual consumptions and greenhouse gas emissions. The end-of-life stage is more challenging due to the specificity of the material, since PA6 is rarely recycled from textiles, sportswear, or automotive applications. With an increase in the share of U.S. consumption of synthetic fibers and only about 5% of total polyamide waste being recycled, degradation and upcycling technologies that divert these materials from landfills need to be developed. Thus, development of novel biological mechanisms, enzymes, and pathways for deconstruction of PA6 is crucial to reduce its environmental impact and increase circularity in the global plastics economy. Currently, little understanding on PA6 biological degradation exists, and no direct enzymatic degradation pathways have been identified. PA6 is a highly unique polymer with structural peculiarities that make degradation enzyme development significantly more difficult than for other plastics, such as polyesters, that have demonstrated early success in this emerging field.

### SUMMARY OF THE INVENTION

The present invention relates to enzymatic degradation of polyamides. The present invention provides novel ways of degrading polyamides including Nylon 6 and Nylon 6,6 (polycaprolactam). Microorganisms produce enzyme(s) useful for degrading polyamides. This enzyme or enzymes can be combined with known manganese peroxidase, proteases, subtilisins, and/or cutinases to provide compositions and methods for degrading polyamides. In some embodiments, microbial cultures of Candida Antarctica are used to produce proteases and lipases; in some embodiments, Ideonella sakaiensis species are used to produce cutinases;

however, the invention is not limited to particular microorganisms.

In another aspect, the invention may include (1) obtaining/providing a composition mixture containing one or more microorganisms capable of producing enzymes that will degrade polyamides; (2) isolating and enriching one or more microorganisms that exhibits high levels of expression of polyamide-degrading enzymes; and (3) producing enzymes using a strong constitutive promoter that results in overexpression of protein and secretion into an extracellular medium. In some preferred embodiments, the composition mixture is obtained from a landfill or other waste storage site. In some embodiments, the microorganisms comprise Ganoderma sp., P. chrysosporium, P. radiata, Anoxybacillus, Trametes versicolor, Fusarium solani pisi, Bjerkandra adusta, Cerenna unicolor, and combinations thereof. Preferred polyamides comprise nylon 6 and nylon 6,6. In some embodiments, the enzymes comprise manganese peroxidase, proteases, subtilisins, and/or cutinases.

In another aspect, the invention provides a method of decomposing a polyamide, comprising: providing a composition comprising a polyamide; treating the polyamide with an enzyme composition comprising one or more of manganese peroxidase, cutinase, protease, and subtilisin. Preferably, the enzyme composition comprises one or more of manganese peroxidase, protease and subtilisin. In another preferred embodiments, the enzyme composition comprises subtilisin and an enzyme originating from a fungus. In some embodiments, the enzyme composition comprises subtilisin and one or more of manganese peroxidase, protease and cutinase. In some embodiments, the polyamide is physically disrupted such as by grinding or bending. Preferably, physical disruption occurs prior to (and/or during) the step of treating with an enzyme.

In a further aspect, the invention method of supplementing polyamide degradation by fungal cultures, comprising: providing a polyamide source with a polyamide degrading fungal culture; adding cell-free enzyme extracts to enhance cellular growth on and utilization of a polyamide; wherein the cell-free enzyme extracts comprise subtilisin, protease and cutinase.

Preferred fungal cultures include Ganoderma sp., P. chrysosporium, P. radiata, Anoxybacillus, Trametes versicolor, Fusarium solani pisi, Bjerkandra adusta, Cerenna unicolor and combinations thereof. By supplementing cell-free enzyme extract to a growing culture, the supplemented enzymes can create polyamide degradation products (such as oligomers and monomers) for the growing culture to consume/metabolize until the cells create more of their own polyamide-degrading enzyme production. For manganese peroxidase, MnCl2 and H2O2 can be added.

In another aspect, the method provides a method of supplementing polyamide degradation by fungal cultures, comprising: providing a polyamide source with a polyamide degrading fungal culture; and combining veratryl alcohol with the polyamide source with polyamide degrading fungal cultures.

In a further aspect, the invention provides a method degrading a polyamide (preferably a polycaprolactam such as nylon6, or nylon6,6), comprising: treating a composition comprising nylon6 by the with lipase or cutinase; and maintaining contact of the nylon6 with the lipase of cutinase for at least one hour.

Any of the aspects of the invention can be further characterized by one or any combination of the following: wherein the contact is maintained for at least one day, or for at least a week, or between one and ten days; wherein the is treated with lipase and cutinase; wherein the polyamide is physically disrupted; wherein the polyamide is treated with cutinase; wherein the polyamide is degraded by at least twice the amount, or between 2 and 4 times the amount, as measured by loss of mass, as compared to an identical treatment without an added enzyme; wherein the polyamide is in the form of a film and is treated with lipase; wherein the polyamide is degraded by at least 30% more or at least 50% more, or between 30 and 100% more of the amount, as measured by loss of mass or film thickness, as compared to an identical treatment without an added enzyme.

In some aspects, the invention provides isolation and enrichment of the appropriate fungus, followed by laccase production using a strong constitutive promoter results in overexpression of protein and secretion into an extracellular medium. Additional application of subtilisin and cutinases could additionally allow for a broader spectrum of substrate specificity and an enhanced degradation of polyamide and degradation byproducts.

### BRIEF DESCRIPTION OF THE FIGURES

FIG. A shows x-ray diffraction results and illustrates a film thickness measurement for monitoring nylon degradation.

FIG. B shows the results of nylon degradation in the presence of enzymes. The results show mass loss for degradation testing of nylon6 powder an nylon6 film.

### EXAMPLES

Degradation of nylon-6 was monitored in the presence of protease, Lipozyme (lipase), cutinase, and manganese peroxidase (MnP). Degradation of Nylon films can be measured by film thickness. Crystallinity monitored by x-ray diffraction. See Fig. A. MnP was optimized using various media recipes to determine the best media candidate for increased MnP concentrations. The greatest degradation of nylon powder was observed for cutinase and nylon film was for lipase. Results are shown in Fig. B.

Elucidation of Best Microorganisms and Enzyme Candidates for Effective PA6 Degradation.

Our research showed promising weight loss of PA6 over time when several candidate strains, consortia, and enzymes were used. For example, 96-hour incubations with cutinases and 1 g of PA6 powder, resulted in 15-23% of mass loss. A commercially available protease displayed 1.75-fold increase in the concentration of 6HA and -3-fold increase in the concentration of caprolactam in the presence of PA6 over 24 hours, with minimal increase in negative controls.

Fungal peroxidases with 4 to 20 units (U) of activity yielded a >30% weight loss in PA6 powder, which was statistically significant when compared with the negative control losses (p <0.05). Based on our experiments, cutinases and fungal peroxidases show promise in PA6 degradation.

We can test both bulk polymer degradation to determine overall transformation efficacy, as well as incubate enzymes and microbial cultures with a series of PA6 oligomers to identify degradation mechanism(s). Strains and enzymes of interest can be grown in the laboratory according to previously developed protocols (24, 25) with PA6 as a sole nitrogen (N) source and glucose to supplement for available carbon. To elucidate effective PA6 degradation with single enzyme preparations, fungal peroxidases can be produced, specifically MnP using overproducing Ganoderma destructans and Cerrena unicolor strains from Battelle's Fungal Archive.

Temperatures and time of incubations can be selected. For example, for cutinase (Thc_Cut1), 45- 50 ° C. is often used to retain good stability and activity of the enzyme. In addition, a higher temperature would have a potential to increase the mobility of polymer chains, which has been proven to increase the enzymatic polymer hydrolysis. To define kinetic parameters of target enzymes in the presence of PA6, we can use Michaelis-Menten Kₘ and Vₘₐₓ, at 25° C. and pH=7. The calculations to establish corresponding values will be made with “Origin” software. MnP activity will be measured at 270 nm through the change in formation of Mn (III)-malonate complexes (Ε270=11,590 M-1 cm-1) with H202. Similarly, protease can be tested using the ThermoFisher Protease Assay and cutinase activity assessed at 410 nm through the production of p-nitrophenyl from p-nitrophenylbutyrate (26). One unit (U) of enzyme activity will be defined as 1 μmol of substrate oxidized per minute.

The ratio of enzyme loading to the PA6 feedstock (polymer or oligomers) can be estimated prior to the laboratory experimentation. After reactions are stopped, changes in bulk polymer molecular weight distribution can be identified using GPC. LC-MS and NMR can be used to detect changes in monomers and oligomers in liquid samples. Additional analytical details are listed below. Based on the mechanistic understanding gained here, enzymes capable of transforming different fractions of PA6 polymer or oligomers may be combined for synergistic effects.

Analytical Methods for PA6 Degradation Validation.

LC-MS can be used by to quantify PA6 monomers and oligomers to enable an understanding of degradation products generated upon enzymatic reaction. ¹³C NMR can be used to detect changes in functional groups of both bulk polymer and oligomers. Size exclusion chromatography (SEC) and/or GPC can be used to investigate the bulk PA6 polymer changes during degradation. The degradation of the PA6 substrate can be evaluated using size exclusion chromatography coupled with multi-angle light scattering (SEC-MALS), which allows for quantitative polymer molecular weight analysis using established literature procedures. In this approach, 1-1-1,3,3,3-hexafluoroisopropyl-alcohol (HFIP) is used as solvent since it can dissolve nylons under ambient conditions. For analysis, the polymer sample can be chromatographically separated by size using a series of three PL-gel columns designed for HFIP and available from Agilent. Quantitative analysis can be performed through dual MALS and refractive index detectors purchased from Wyatt Technologies, which provide absolute weight average molecular weight (M_{w}) and sample concentrations, respectively.

The extent of polymer degradation will be evaluated qualitatively through changes in chromatographic distributions and peak retention times. Degradation products will have a lower molecular weight relative to the nylon starting material and will appear at longer retention times. Some method development may include the addition of NaTFA salts to prevent polymer aggregation and optimizations to minimize error in quantitation. The dn/dc of the starting material can be used for accurate quantitation and can be measured through batch injections of known concentrations of PA6 dissolved in HFIP into a refractometer.

Microbial Testing

Anoxybacillus rupiensis, Trametes versicolor, Bjerkandra adusta, and Phanaerochaete chrysosporium can be assessed for degradation of nylon under N-limited conditions. An enzyme stimulation approach can be used for Ganoderma sp., Cerenna unicolor, and Fusarium solani subsp. pisi.

Fungal Isolate Testing

Laccase, manganese peroxidase, and lignin peroxidase enzyme production in and Cerenna unicolor

Cerenna unicolor has previously been documented to produce laccase, lignin peroxidase, and manganese peroxidase. These strains can be tested for nylon-6 degradation. First, media conditions can be assessed to produce optimal concentrations of manganese peroxidase (Table 1). This will be performed in 50 mL vented conical vials with 5% strain transfers. All additives can be included in media formulations and pH testing performed for MnP.

TABLE 1
Media recipes and additives for manganese peroxidase
production optimization

| Media | Recipe | | Additive |
|---|---|---|---|
| Mandarin Peel | 1 | g/L KH2PO4, | None |
| Medium | 0.5 | g/L MgSO4, | |
| | 0.1 | g/L CaCl2, | |
| | 0.005 | g/L FeSO4*7H2O, | |
| | 1 | g/L peptone, | |
| | 2 | g/L yeast extract, | |
| | 10 | g/L glycerol, | |
| | 20 | g/L mandarin peels | |
| Mandarin Peel | | | 30.2 mg MnSO4*H2O |
| Medium + MnSO4 | | | |
| Mandarin Peel | | | 3.774 mL Tween-80 |
| Medium + Tween 80 | | | per 1 L media |
| Mandarin Peel | | | 30.2 mg MnSO4*H2O, |
| Medium + MnSO4 + | | | 3.774 mL Tween-80 |
| Tween 80 | | | per 1 L media |
| Synthetic Medium | 1 | g/L KH2PO4, | None |
| with Glycerol | 0.5 | g/L MgSO4, | |
| | 0.1 | g/L CaCl2, | |
| | 0.005 | g/L FeSO4*7H2O, | |
| | 2 | g/L peptone, | |
| | 2 | g/L yeast extract, | |
| | 10 | g/L glycerol | |
| Synthetic Medium | | | 30.2 mg MnSO4*H2O |
| with Glycerol + | | | |
| MnSO4 | | | |
| Synthetic Medium | | | 3.774 mL Tween-80 |
| with Glycerol + | | | per 1 L media |
| Tween 80 | | | |
| Synthetic Medium | | | 30.2 mg MnSO4*H2O, |
| with Glycerol + | | | 3.774 mL Tween-80 |
| MnSO4 + Tween 80 | | | per 1 L media |
| Glucose Medium | 20 | g/L glucose, | None |
| | 10 | g/L yeast extract, | |
| | 2 | g/L KH2PO4, | |
| | 0.1 | g/L CuSO4*5H2O | |
| Glucose Medium + | | | 30.2 mg MnSO4*H2O |
| MnSO4 | | | |
| Glucose Medium + | | | 3.774 mL Tween-80 |
| Tween 80 | | | per 1 L media |
| Glucose Medium + | | | 30.2 mg MnSO4*H2O, |
| MnSO4 + Tween 80 | | | 3.774 mL Tween-80 |
| | | | per 1 L media |

Manganese peroxidase expression for nylon-6 degradation

Using the best results of the media optimization, Ganoderma sp. and Cerenna unicolor can be cultivated in the presence of nylon-6 powder (0.5%). Aliquots taken daily for 1 week to assess MnP activities, amine concentrations, and LC-MS.

Cutinase enzyme production in F. solani pisi

Onboarding F. solani pisi for nylon-6 testing

F. solani pisi ATCC 200576 can be onboarded as a frozen culture from the ATCC and stored in the -80 ° C. after use. A 3-phase streak plate can be performed on V8 juice agar, stored at 27 ° C. Once colonies have grown, a single colony can be inoculated into V8 juice broth and allowed to grow prior to experimentation at 27 ° C. and 140 rpm shaking. At this point, glycerol stocks can be made in triplicate.

Expression of cutinase for nylon-6 degradation

F. solani pisi can be transferred to a basal mineral medium with 1% flaxseed oil (w/v) as the carbon source and incubated at 27° C. and 140 rpm shaking. and allowed to acclimate for at least one generation. Nylon-6 powder (0.5%) can be added to cultures for degradation testing. Aliquots can be taken daily for 1 week to assess enzyme activities (cutinase), amine concentrations, and LC-MS.

Nylon-6 as a sole nitrogen source in P. chrvsosporium, T. versicolor, and B. adusta

Onboarding fungal strains for nylon-6 testing

P. chrysosporiurn DSMZ 6909 can be onboarded as an actively growing culture from the DSMZ. A 3-phase streak plate will be performed on Malt Extract Peptone Agar, stored at 27° C., and glycerol stock made in triplicate. Once colonies have grown, a single colony can be inoculated into

Malt Extract Peptone Broth and allowed to grow prior to experimentation at 27 ° C. with 140 rpm shaking.

T. versicolor DSMZ 6401 can be onboarded as an actively growing culture from the DSMZ. A 3-phase streak plate will be performed on Malt Extract Peptone Agar, stored at 27 ° C., and glycerol stock made in triplicate. Once colonies have grown, a single colony can be inoculated into Malt

Extract Peptone Broth and allowed to grow prior to experimentation at 27 ° C. with 140 rpm shaking.

B. adusta ATCC 62238 can be onboarded as a frozen culture from the ATCC and stored in the -80° C. after use. A 3-phase streak plate can be performed on Potato Dextrose Agar and stored at 27° C. Once colonies have grown, a single colony can be inoculated into Malt Extract Peptone Broth and allowed to grow prior to experimentation at 27 ° C. and 140 rpm shaking. At this point, glycerol stocks will be made in triplicate.

Nylon as a sole N source

All cultures will be acclimated to basal glucose medium prior to testing for nylon-6 degradation. Nylon-6 (0.5% powder) will be added at 27 ° C. and 140 rpm shaking.

Aliquots will be taken every other day for 14 days to assess OD, amine concentrations (ninhydrin assay), byproduct concentrations (LC-MS), and for RNA extractions (RNAseq).

The following strains can be obtained in conjunction with a USDA permit.
1. Trametes versicolor
2. Fusarium solani subsp. Pisi
3. Bjerkandra adusta
4. Cerenna unicolor

Fungal enzyme testing

The efficacy of manganese peroxidase (MnP), laccase (Lac), and lignin peroxidase (LiP) on bulk nylon-6 polymers can be tested. While lignin peroxidase and laccase have not been previously implicated in nylon-6 transformation, other fungal species have been able to grow on bulk nylon-6 polymers with unknown mechanisms of degradation, making it plausible these enzymes may be able to transform nylon-6. Further, manganese peroxidase has been identified as transforming nylon-6 through radical species generation, a process that also occurs with lignin peroxidase. Nylon powder (50μm, 0.25 g) and film (0.025 mm, cut into 1 in. x 2 in. strips) can be tested. Nylon powder and films can be added to 50 mL conical tubes as required by experiments, with 5 mL of MilliQ water. Then tubes can be sonicated for 5 minutes, then decanted and resuspended in autoclaved medium (all done within a biosafety cabinet).

Enzymatic formulations can be made with set amounts of enzyme powder (see Table 1) and 25 mL 0.05 M sodium acetate buffer at pH 5.5. For initial screening of fungal enzymes, enzymes can be tested on nylon powder, aliquots will be taken every 2 hours for the first 12 hours, then at 24 hours. Enzyme concentrations used in Table 1 may be updated depending on refining of Ganoderma sp. and Cerenna unicolor enzyme production. Enzyme activity can be assessed. Ninhydrin assay can be performed to identify release of amines during nylon-6 degradation, using t=0 and t=24. Samples from t=0, t=12 t=24 will be stored at -20 ° C. for LC-MS analysis.

TABLE 2
Setup of nylon-6 testing with fungal enzymes.

| Sample Name | Enzyme volume added | Replicates |
|---|---|---|
| Negative Control | 0 | 1 |
| Lac 2000 U | 1.276 mL | 2 |
| MnP 400 U | To be determined | 2 |
| LiP 2000 U | To be determined | 2 |
| Lac & MnP (2000 & 400 U) | 1.276 mL | 2 |
| Lac & LiP (2000 & 2000 U) | | 2 |
| MnP & LiP (400 & 2000 U) | | 2 |

Subtilisin enzyme testing

Subtilisin is a protease capable to alter surface properties of nylon-6. Mechanistically, this enzyme cleaves amide bonds, and may be capable of cleaving the main amide bond in nylon-6. Subtilisin was originally isolated in Bacillus. Coincidentally, B. cereus is one of the few bacterial species that has shown promise in degrading nylon-6 (Sudhakar et al., 2007).

In the same manner as the enzymes, subtilisin can be tested for nylon-6 powder degradation. Film degradation can be tested. Subtilisin solutions can be made with set amounts of enzyme solution (see Table 2) and 25 mL 0.05 M sodium acetate buffer at pH 8.5, incubated at 55-60 C. The enzyme activity will be assayed using a 96-well plate colorimetric method (PierceTM Colorimetric Protease Assay Kit, ThermoFisher Scientific, Section 2.6.1). Ninhydrin assay can be performed to identify release of amines during nylon-6 degradation, using t=0 h and t=24 h. Samples from t=0 h, t=12 h t=24 h will be stored at -20 ° C. for LC-MS analysis.

TABLE 3
Setup of nylon-6 testing with subtilisin.

| | Enzyme liquid | |
|---|---|---|
| Sample Name | added (mL) | Replicates |
| Negative Control-Powder | 0 | 2 |
| Negative Control-Film | 0 | 2 |
| Subtilisin 1220 U-Powder | 0.5 mL | 3 |
| Subtilisin 1220 U-Film | 0.5 mL | 3 |
| Subtilisin 2440 U-Powder | 1 mL | 3 |
| Subtilisin 2440 U-Film | 1 mL | 3 |

Cutinase enzyme testing

Cutinases are a class of lipases characterized by their ability to degrade cutin present in plant cuticles. Due to the high hydrophobicity of nylon-6, an enzyme like cutinase tailored to degrade hydrophobic compounds may be a valuable strategy. Further, these enzymes have shown promise in degrading PET polymers (Carneil et al., 2017). Commercially available enzyme formulations include Novozyme 51032 (HiC) and Lipozyme CALB (CALB). HiC produced by E. coli with a T. fusca cutinase gene can also be used.

In the same manner as the enzymes in 2.1 and 2.2, cutinases can be tested on nylon-6 powder and film. HiC, CALB, and 50:50 HiC:CALB can be tested. Cutinase solutions can be made with set amounts of enzyme solution (see Table 3) and 25 mL 200 mM sodium phosphate buffer at pH 7.0, incubated at 50 ° C. Enzyme activity can be assayed.

TABLE 4
Setup of nylon-6 testing with cutinase.

| | Enzyme liquid | | |
|---|---|---|---|
| Sample Name | added (mL) | | Replicates |
| Negative Control-Powder | 0 | | 2 |
| Negative Control-Film | 0 | | 2 |
| HiC 7800 U-Powder | 0.5 | mL | 3 |
| HiC 7800 U-Film | 0.5 | mL | 3 |
| CALB 6000 U-Powder | 1 | mL | 3 |
| CALB 6000 U-Film | 1 | mL | 3 |
| HiC 7800 U + CALB | 0.5 | mL HiC, | 3 |
| 6000 U-Powder | 1 | mL CALB | |
| HiC 7800 U + CALB | 0.5 | mL HiC, | 3 |
| 6000 U-Powder | 1 | mL CALB | |

1-3. (canceled)

4. A method of decomposing a polyamide, comprising:
providing a composition comprising a polyamide;
treating the polyamide with an enzyme composition comprising one or more of manganese peroxidase, protease, lipase, cutinase, and subtilisin.

5. The method of claim 4 wherein the polyamide comprises nylon6 or nylon6,6.

6. The method of claim 5 wherein the nylon6 or nylon6,6 is treated with lipase, cutinase, or a combination of lipase and cutinase.

7. The method of claim 5 wherein the enzyme composition comprises two or more of manganese peroxidase, protease , and subtilisin.

8. The method of claim 5 wherein the enzyme composition comprises subtilisin and an enzyme originating from a fungus.

9. The method of claim 5 wherein the enzyme composition comprises subtilisin and one or more of manganese peroxidase, and cutinase.

10. The method of claim 5 wherein the polyamide is physically disrupted such as by grinding or bending.

11. A method of supplementing polyamide degradation by fungal cultures, comprising:
providing a polyamide source with a polyamide degrading fungal culture;
adding cell-free enzyme extracts to enhance cellular growth on and utilization of a polyamide;
wherein the cell-free enzyme extracts comprise subtilisin and cutinase.

12. The method of claim 11 wherein the fungal cultures comprise Ganoderma sp., P. chrysosporium, P. radiata, Trametes versicolor, Fusarium solani pisi cutinase, Bjerkandra adusta, Cerenna unicolor, and combinations thereof.

13. The method of claim 11 wherein the polyamide comprises nylon6 or nylon66.

14. The method of claim 13 wherein veratryl alcohol is added to further enhance degradation.

15-16. (canceled)

17. A method degrading nylon6, comprising:
treating a composition comprising nylon6 by the with lipase or cutinase; and
maintaining contact of the nylon6 with the lipase of cutinase for at least one hour.

18. The method of claim 17 wherein the contact is maintained for at least one day.

19. The method of claim 18 the nylon6 is treated with lipase and cutinase.

20. The method of claim 18 wherein the nylon is physically disrupted and the nylon6 is treated with cutinase.

21. The method of claim 20 wherein the nylon is degraded by at least twice the amount, or between 2 and 4 times the amount, as measured by loss of mass, as compared to an identical treatment without an added enzyme.

22. The method of claim 18 wherein the nylon6 is in the form of a film and is treated with lipase.

23. The method of claim 22 wherein the nylon is degraded by at least 30% more or at least 50% more, or between 30 and 100% more of the amount, as measured by loss of mass or film thickness, as compared to an identical treatment without an added enzyme.
